(19) United States
(12) Patent Application Publication
(10) Pub. No.: US 2009/0105961 A1
(43) Pub. Date: Apr. 23, 2009

(54) METHODS OF NUCLEIC ACID IDENTIFICATION IN LARGE-SCALE SEQUENCING

(75) Inventor: Radoje Drmanac, Los Altos Hills, CA (US)

Correspondence Address: MORGAN, LEWIS & BOCKIUS, LLP, ONE MARKET SPEAR STREET TOWER, SAN FRANCISCO, CA, 94105, US

(73) Assignee: COMPLETE GENOMICS, INC., Mountain View, CA (US)

(21) Appl. No.: 11/938,213

(22) Filed: Nov. 9, 2007

Related U.S. Application Data
(60) Provisional application No. 60/864,993, filed on Nov. 9, 2006.

(51) Int. Cl. G01N 33/48

(52) U.S. Cl. 702/20

(57) ABSTRACT

The present invention provides methods for determining a base probability in a target nucleic acid within an experimental data set. The methods of the invention provide specific methods of improving accuracy of base calling for experimental sequencing data compared to conventional methods. The experimental base values used in the methods of the present invention provide relative base probabilities within an experimental data set that are robust and uniformly optimal regardless of the experimental conditions.

### CROSS-REFERENCE TO RELATED APPLICATIONS

This application claims priority to provisional application Ser. No. 60/864,993, filed Nov. 9, 2006, which is hereby incorporated by reference in its entirety.

### FIELD OF THE INVENTION

This invention relates to a present invention relates to methods for evaluating and comparing biological sequences. In particular, the invention provides improved methods for identifying individual nucleic acids in large target sequences.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The computational complexity involved in sequence analysis of three billion base pairs in the human genome is further compounded by the accuracy requirements of clinical diagnostics such that 60 billion or more sequence data points must be analyzed to provide one accurate genome sequence read. This complexity was dealt with in early sequencing methods by generating sequence data from thousands of isolated, very long fragments of DNA, thereby preserving the contextual integrity of the sequence information and reducing the redundant testing required for accurate data. However, this approach, used to generate the first complete human genome, cost hundreds of millions of dollars per genome due to the up-front complexity of preparing the genome fragments and the relative high cost of many individual biochemical tests.

In addition, contextual information in the genome is compounded by the presence of two distinct copies of the genome in each human cell such that accurate clinical analysis and diagnosis requires the ability to distinguish DNA sequence as a function of genome copy, more commonly referred to as the genome “haplotype”. Thus, a major challenge is to distinguish sequence differences between the two unique copies of the three billion DNA bases interspersed with millions of inherited single nucleotide polymorphisms (SNPs), hundreds of thousands of short insertions and deletions and hundreds of spontaneous mutations.

Recently, specific programs have been developed that aid in the identification of a single nucleotide polymorphism (“SNP”) within a complete DNA sequence, and to aid in the confidence of the identification based on comparison of the sequence with reference sequences or multiple different copies of the sequence. This identification of SNPs and validation is based on different sets of samples, and the data used in such programs is error-prone and known to harbor artifactual apparent polymorphisms. There is thus a need for improved nucleotide identification based primarily on experimental information.

### SUMMARY OF THE INVENTION

The present invention provides methods for determining relative base probabilities in a set of target nucleic acids using an experimental data set. The methods of the invention provide specific methods of improving accuracy of base calling for experimental sequencing data compared to conventional methods. Furthermore, the invention provides methods for accurate determination of measurements that estimate the likelihood that a base is present at a position in a target nucleic acid. The experimental base values used in the methods of the present invention provide information to determine relative base probabilities within an experimental data set that are robust and uniformly optimal regardless of the variation in experimental conditions. The relative base probabilities assist in accurate determination of error rates in base calling, e.g., in one or more targets nucleic acids from a genome, and determining probabilities and error rates of a called base in the genome. Such probabilities can be used alone or in combination with known or expected polymorphism and/or mutation.

In one aspect of the invention, a method is provided for determining a relative base probability, the method comprising: providing a statistically significant number of experimental base values for a set of target nucleic acids; creating a distribution of said experimental base values; determining a relative base probability of a base at a position of a target nucleic acid by comparing its experimental base value with the distribution of experimental base values.

In specific aspects of the embodiments of the invention, the relative base probability of a base at a position can be used to “call”, or identify, the base at that position, e.g., for use in assembly of the target nucleic acid sequence, e.g. assembly of a genome a sample.

Experimental base values can, in certain aspects, be obtained for a position in a target nucleic acid by identifying the position relative to a priming site or adaptor binding site used in sequencing the target nucleic acid. Multiple experimental base values for one or each four bases for a position in a target nucleic acid can be used in the creation of a distribution of the base values.

In very specific aspects, the experimental base values used for a given distribution are obtained in a single sequencing experiment. In another aspect, the experimental base values are obtained in two or more sequencing experiments using substantially the same conditions and a substantially similar target nucleic acid.

In specific aspects of the invention, the raw data generated from the sequencing experiment is adjusted prior to the creation of the distributions to provide the most accurate use of the experimental data, e.g., by discarding data with very low confidence or data from portions of the sequencing experiment with known experimental error. In specific aspects, the experimental base values are normalized prior to the creation of the distributions of the invention. In another aspect, the invention provides a method for determining relative base probabilities in a target nucleic acid, comprising: providing experimental base values for a base at a position in set of target nucleic acids; dividing said base values into two or more groups according to associated experimental measurements, wherein each group comprises a statistically significant number of experimental base values; creating a distribution of said bases values for each group; and determining the relative base probability of a base in a position of a target nucleic by comparing its experimental base value with the distribution of experimental base values in the relevant group. In this context, a “relevant” group for purposes of comparison refers to the group of experimental base values in which the base is included.

In one aspect of the invention, the invention provides methods of determining a relative base probability a base at a position in a target nucleic acid, comprising the steps of: obtaining a plurality of experimental intensity base values for a statistically significant number of nucleotides at a position within a nucleic acid; creating a base intensity distribution for this position based on the plurality of base intensity values obtained from the sequencing experiment; and comparing the base intensity value of a base at a position in a target nucleic acid to the signal intensity distribution for this position within the target nucleic acid. In this specific aspect of the invention.

In another aspect of the invention, the invention provides methods of determining a relative base probability of a first base at a position in a target nucleic acid comprising the steps of obtaining a plurality of experimental intensity base values at a position in a target nucleic acid; dividing the experimental intensity values into groups based on the identification of a second base with a known position relative to the first base; creating an intensity value distribution for each group based on the plurality of base values obtained, wherein the groups comprise statistically significant number of experimental intensity values; and comparing the experimental intensity value of the first base to the distribution created from a relevant group to determine a relative base probability. In this context, a “relevant” group for purposes of comparison refers to the group of experimental intensity values in which the first base is included.

In yet another aspect of the invention, the invention provides methods of identifying a relative base probability for the calling of an individual nucleotide in a sequencing experiment comprising the steps of obtaining individual intensities for a statistically significant number of interrogated nucleotides within a sequencing experiment; categorizing the individual intensities based on the identification of a second nucleotide in a defined position with respect to the interrogated nucleotide; comparing the signal intensity to a signal intensity distribution previously created using data created under substantially similar experimental conditions, e.g., data from a prior experiment using substantially the same conditions and the same or a similar target nucleic acid.

In a specific aspect, the invention comprises a computer program product that calculates relative base probabilities from experimental base values, comprising: computer code that receives a plurality of signals corresponding to a statistically significant number of experimental base values for a target nucleic acid; computer code for creating a distribution of said experimental base values; computer code for determining a relative base probability of a base at a position of the target nucleic acid by comparing its experimental base value with the distribution of experimental base values; and a computer readable medium that stores said computer codes. This product optionally provides computer code to generates a base call for the base at a position in a target nucleic acid.

In another aspect, the invention provides a system to determine relative base probabilities, comprising: 1) a processor; and 2) a computer readable medium coupled to said processor for storing a computer program comprising: computer code that receives a plurality of signals corresponding to a statistically significant number of experimental base values for a target nucleic acid; computer code for creating a distribution of said experimental base values; And computer code for determining a relative base probability of a base at a position of the target nucleic acid by comparing its experimental base value with the distribution of experimental base values. This system optionally also comprises computer code that generates a base call for the base at a position in a target nucleic acid.

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

### BRIEF DESCRIPTION OF THE FIGURES

The following drawings are representational of one format for presentation of the data provided from implementation of the invention. These drawings are not intended to limit in any way the implementation of aspects of the invention as described herein, but rather to aid in clarification of the underlying concepts of the invention.

FIG. 1 is an exemplary, representative graph illustrating subdivisions of the four experimental base values for experimental base values for a specific position within a target nucleic acid.

FIG. 2 is an exemplary, representative graph illustrating the distributions of the experimental base values for a specific position within a sequencing experiment, wherein the experimental base value distribution is provided in two groups for each potential nucleotide position.

FIG. 3 is an exemplary, representative graph illustrating the distributions of experimental base values for a detection of a single base at a specific position within a defined position context in a target nucleic acid.

FIG. 4 is an exemplary, representative graph illustrating the distributions of the experimental base values for a base in a specific position in a target nucleic acid, and use of these distributions in identifying a relative base probability.

FIG. 5 shows an intensity graph comparing the experimental base intensity values of base C and base A at a specific position of a target nucleic acid.

FIG. 6 illustrates a computer system for use with the present invention

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach”1984, IRL Press, London; Nelson and Cox (2000), Lehninger, Principles of Biochemistry 3^{rd} Ed., W.H. Freeman Pub., New York, N.Y.; and Berg et al. (2002) Biochemistry, 5th Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a target nucleic acid” refers to one or multiple copies of such, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

An “associated experimental measurement” as used herein refers to the identity and/or position of one or more other nucleotides within a target nucleic acid relative to a base to be interrogated, the quantity of target nucleic acid analyzed in any given experiment or subset of an experiment, the specific base content (i.e., percentage of specific nucleotides) in the target nucleic acid being analyzed, and the like.

“Experimental base value” as used herein refers to a value derived from a sequencing experiment that is indicative of the presence of a specific base at a specific position in a target nucleic acid. For example, in interrogating a base at a specific position in a DNA fragment, four base values will be identified—one for each potential nucleotide. Experimental base values can be experimental intensity base values, or any other measurable indicator of a specific base at a specific position in a target nucleic acid.

“Experimental intensity base values” and “Experimental intensity values” are experimental base values created by identification of a signal intensity specific to the presence of a particular nucleotide at a position in a target nucleic acid. Examples of experimental intensity base values include base values created by the hybridization of a fluorescently-labeled probe that hybridizes to a specific nucleotide, by the incorporation of a labeled dNTP at a specific position in a target nucleic acid, and the like.

“Complementary” or “substantially complementary” refers to the hybridization or base pairing or the formation of a duplex between nucleotides or nucleic acids, such as, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the other strand, usually at least about 90% to about 95%, and even about 98% to about 100%.

“Hybridization” refers to the process in which two single-stranded polynucleotides bind non-covalently to form a stable double-stranded polynucleotide. The resulting (usually) double-stranded polynucleotide is a “hybrid” or “duplex.” “Hybridization conditions” will typically include salt concentrations of less than about 1M, more usually less than about 500 mM and may be less than about 200 mM. A “hybridization buffer” is a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are usually performed under stringent conditions, i.e., conditions under which a probe will hybridize to its target subsequence but will not hybridize to the other, uncomplimentary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. Exemplary stringent conditions include a salt concentration of at least 0.01M to no more than 1M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of 30° C. are suitable for allele-specific probe hybridizations.

“Ligation” means to form a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide. Template driven ligation reactions are described in the following references: U.S. Pat. Nos. 4,883,750; 5,476,930; 5,593,826; and 5,871,921.

The term “signal intensity” will generally refer to the intensity of a detectable reaction providing information on the likelihood that a nucleotide at a defined position contains a specific base. Examples of such identifying reactions include, but are not limited to, labeled probe hybridization reactions, labeled probe-ligation reactions, nucleotide synthesis with labeled nucleotides, and the like. For naturally-occurring DNA, a signal intensity is generally determined four times at each nucleotide position, one for each of the four naturally-occurring bases.

The term “target nucleic acid” as used herein means a nucleic acid sequence from a gene, a regulatory element, genomic DNA, cDNA, RNAs including mRNAs, rRNAs, siRNAs, miRNAs and the like, or a fragment thereof. A target nucleic acid may be a target isolated from a sample, or a secondary target such as a product of an amplification reaction or a fragment of one of these. In a specific aspect of the invention, the target nucleic acid can be obtained from a sample comprising an entire genome, more specifically an entire mammalian genome, even more specifically an entire human genome. In other specific aspects, the target nucleic acid is a specific fragment from a complete genome.

The terms “base” when used in the context of identification refers to the purine or pyrimidine group (or an analog or variant thereof) that is associated with a nucleotide at a given position within a target nucleic acid. Thus, to call a base or to identify a nucleotide both refer to the identification of the purine or pyrimidine group (or an analog or variant thereof) at a specific position within a target nucleic acid.

“Nucleic acid”, “oligonucleotide”, or grammatical equivalents used herein refer generally to at least two nucleotides covalently linked together. A nucleic acid generally will contain phosphodiester bonds, although in some cases nucleic acid analogs may be included that have alternative backbones such as phosphoramidite, phosphorodithioate, or methylphosphoroamidite linkages; or peptide nucleic acid backbones and linkages. Other analog nucleic acids include those with bicyclic structures including locked nucleic acids, positive backbones, non-ionic backbones and non-ribose backbones. Modifications of the ribose-phosphate backbone may be done to increase the stability of the molecules; for example, PNA:DNA hybrids can exhibit higher stability in some environments.

The term “sequencing experiment” as used herein refers to one or a series of biochemistry sequencing reactions to identify undetermined sequences in a target nucleic acid or a fragment thereof. A sequencing reaction, when it includes several reactions, is generally performed under substantially same conditions and on like nucleic acids, e.g., fragments of a single human genome.

“Probe” means generally an oligonucleotide that is complementary to a target nucleic acid under investigation. Probes used in certain aspects of the claimed invention are labeled in a way that permits detection, e.g., with a fluorescent or other optically-discernable tag.

### DETAILED DESCRIPTION OF THE INVENTION

The description of the following aspects of the various embodiments of the invention primarily relate to identification of a single base in a target nucleic acid at a specific position. The invention also related to identification of two or more bases experimentally, depending upon the experimental approach of the identification of the experimental base values provided for use in the present invention.

### THE INVENTION IN GENERAL

The ability to achieve high accuracy in the calling of assembled bases to identify the sequence of a target nucleic acid requires accurate assessment of the confidence or calling of individual raw base calls. This is especially important for assembly of experimental data resulting from high-throughput screening approaches, where the sheer volume of the data and experimental variability can increase the likelihood of sequencing errors or background noise, and the assembly of sequence of long stretches of nucleic acids requires the identification of specific sequences within the greater context of the target nucleic acid. Furthermore, an accurate assessment of raw data allows higher accuracy of the assembled sequence using fewer reads per base in the assembly process, thus reducing the cost of the assay. Assembled sequence with high accuracy and accurately estimated confidence levels and/or error rates is especially critical for genetic diagnostics.

In specific aspects, methods of the invention provide higher probabilities off accurate base calls for each of the four bases at specific positions in a statistically large set of nucleic acid targets analyzed in a sequencing experiment.

Although the disclosure primarily focuses on the use of experimental base values for individual nucleotides within a given target nucleic acid, in a specific aspect of the invention two adjacent nucleotides can be interrogated in the same experimental sequencing reaction. Thus, the methods as described herein are equally applicable for identifying 2-mer or longer base reads experimentally, and using this experimental data in the division into sub-groups and/or the creation of distributions of experimental base values will increase the relative base probabilities of these 2-mer (or more) base reads.

Based on relative base probabilities and base calling of experimental data using the methods of the invention, a preliminary estimate of a target nucleic acid sequences (e.g., when sequencing human genome an individual's “genotype”) can be computed; critically, this initial estimate will generally have fewer mismatches to the individual base calls than did the original reference. Base calling accuracy is then re-estimated based on mismatches to the preliminary individual target nucleic acid sequence, after which the individual target nucleic acid sequence can be re-estimated. In specific aspects of the invention, such a process is re-iterated, and the mapping and base calling confidence estimates will be re-compared to the recalculated sequence estimates as more data is generated and a greater context for each individual nucleotide is determined within the target sequence.

### Obtaining Experimental Base Values

Numerous sequencing experiments can be used with the methods of the present invention to obtain multiple experimental base values corresponding to the presence of a particular base in a defined position in the target nucleic acid. Exemplary methods for obtaining such experimental base values are summarized below, but it will be clear to those skilled in art upon reading the present invention that multiple sequencing approaches can be used with the methods of the invention.

In one specific aspect, the DNA concatamers are used in sequencing by combinatorial probe-anchor ligation reaction (cPAL) (see U.S. Ser. No. 11/679,124, filed Feb. 24, 2007). In brief, cPAL comprises cycling of the following steps: First, an anchor is hybridized to a first adaptor in the DNBs (typically immediately at the 5′ or 3′ end of one of the adaptors). Enzymatic ligation reactions are then performed with the anchor to a fully degenerate probe population of, e.g., 8-mer probes that are labeled, e.g., with fluorescent dyes. At any given cycle, the population of 8-mer probes that is used is structured such that the identity of one or more of its positions is correlated with the identity of the fluorophore attached to that 8-mer probe. For example, when 7-mer sequencing probes are employed, a set of fluorophore-labeled probes for identifying a base immediately adjacent to an interspersed adaptor may have the following structure: 3′F1-NNNNNNAp, 3′-F2-NNNNNNGp. 3′-F3-NNNNNNCp and 3′-F4-NNNNNNTp (where “p” is a phosphate available for ligation). In yet another example, a set of fluorophore-labeled 7-mer probes for identifying a base three bases into a target nucleic acid from an interspersed adaptor may have the following structure: 3′-F1-NNNNANNp, 3′-F2-NNNNGNNp. 3′-F3-NNNNCNNp and 3′-F4-NNNNTNNp. To the extent that the ligase discriminates for complementarity at that queried position, the fluorescent signal provides the identity of that base. In one aspect, one or more fluorescent dyes are used as labels for the oligonucleotide probes. Labeling can also be carried out with quantum dots, as disclosed in the following patents and patent publications, incorporated herein by reference: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like. Commercially available fluorescent nucleotide analogues readily incorporated into the degenerate probes include, for example, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red, the Cy fluorophores, the Alexa Fluor® fluorophores, the BODIPY® fluorophores and the like. FRET tandem fluorophores may also be used. Other suitable labels for detection oligonucleotides may include fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr) or any other suitable label.

Imaging acquisition may be performed by methods known in the art, such as use of the commercial imaging package Metamorph. Data extraction may be performed by a series of binaries written in, e.g., C/C++, and base-calling and read-mapping may be performed by a series of Matlab and Perl scripts. As described above, for each base in a target nucleic acid to be queried (for example, for 12 bases, reading 6 bases in from both the 5′ and 3′ ends of each target nucleic acid portion of each DNB), a hybridization reaction, a ligation reaction, imaging and a primer stripping reaction is performed. To determine the identity of each DNB in an array at a given position, after performing the biological sequencing reactions, each field of view (“frame”) is imaged with four different wavelengths corresponding to the four fluorescent, e.g., 8-mers used. All images from each cycle are saved in a cycle directory, where the number of images is 4× the number of frames (for example, if a four-fluorophore technique is employed). Cycle image data may then be saved into a directory structure organized for downstream processing.

Data extraction for use with this specific approach typically requires two types of image data: bright field images to demarcate the positions of all target nucleic acids in the array; and sets of fluorescence images acquired during each sequencing cycle. The data extraction software identifies all objects with the brightfield images, then for each such object, computes an average fluorescence value for each sequencing cycle. For any given cycle, there are four data-points, corresponding to the four images taken at different wavelengths to query whether that base is an A, G, C or T. These raw base-calls can be used directly in the methods of the invention, or can be subjected to normalization, consolidation or other optimization techniques as described further herein.

In an alternative aspect of the claimed invention, parallel sequencing of the target nucleic acids on a random array is performed by combinatorial sequencing-by-hybridization (cSBH), as disclosed by Drmanac in U.S. Pat. Nos. 6,864,052; 6,309,824; and 6,401,267. In one aspect, first and second sets of oligonucleotide probes are provided, where each set has member probes that comprise oligonucleotides having every possible sequence for the defined length of probes in the set. For example, if a set contains probes of length six, then it contains 4096 (4⁶) probes. In another aspect, first and second sets of oligonucleotide probes comprise probes having selected nucleotide sequences designed to detect selected sets of target polynucleotides. Sequences are determined by hybridizing one probe or pool of probes, hybridizing a second probe or a second pool or probes, ligating probes that form perfectly matched duplexes on their target sequences, identifying those probes that are ligated to obtain sequence information about the target nucleic acid sequence, repeating the steps until all the probes or pools of probes have been hybridized, and determining the nucleotide sequence of the target nucleic acid from the sequence information accumulated during the hybridization and identification processes.

In yet another alternative aspect, parallel sequencing of the target nucleic acids is performed by sequencing-by-synthesis techniques as described in U.S. Pat. Nos. 6,210,891; 6,828,100, 6,833,246; 6,911,345; Margulies, et al. (2005), Nature 437:376-380 and Ronaghi, et al. (1996), Anal. Biochem. 242:84-89. Briefly, modified pyrosequencing, in which nucleotide incorporation is detected by the release of an inorganic pyrophosphate and the generation of photons, is performed on the target nucleic acids in the array using sequences in the adaptors for binding of the primers that are extended in the synthesis.

### Creation of Experimental Base Value Distributions

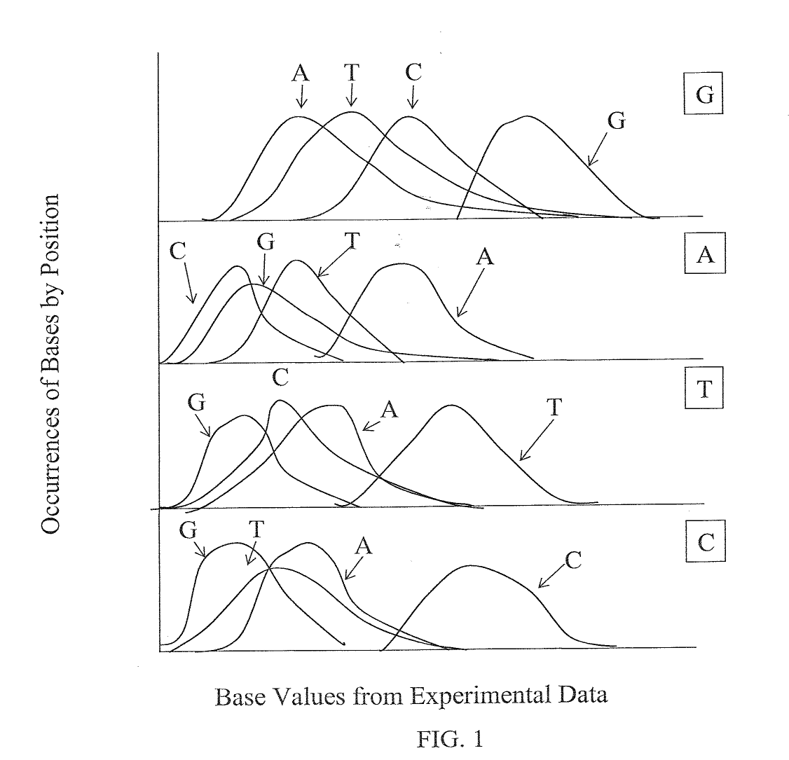

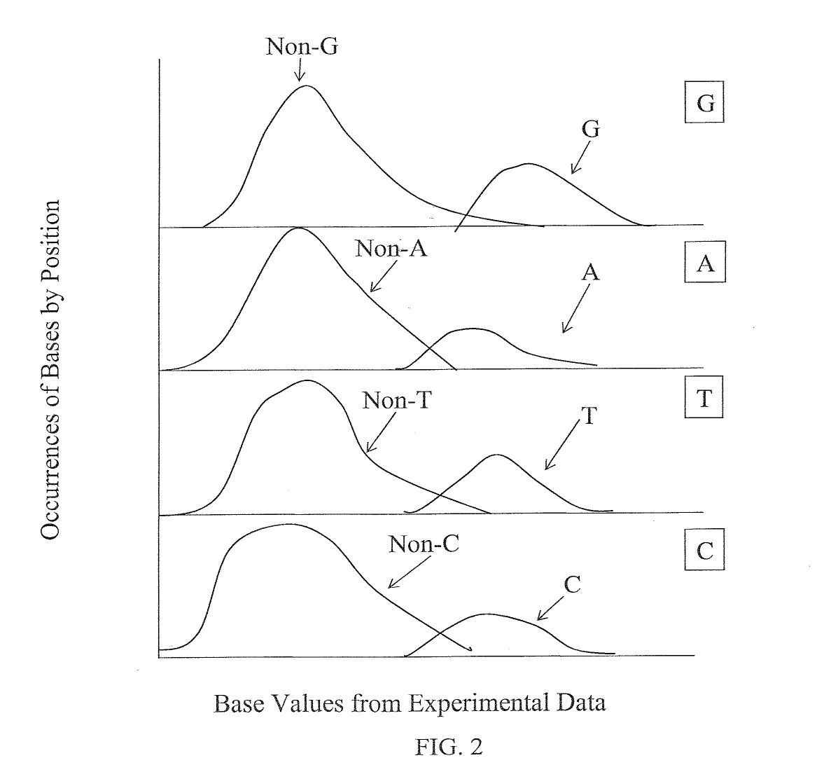

Measurements of experimental base values for interrogated nucleotides are used in the methods of the invention to determine a distribution of the experimental base values for a base at a specific position within a target nucleic acid. In a preferred embodiment, the position is defined by the placement of the base relative to an anchor probe binding site, a primer site for polynucleotide synthesis, or some other discrete sequence provided in the sequencing experiment for the express purpose of identification of the bases in the target nucleic acid. For single base reads there are 4 corresponding measurements (A, T, C, G) for each individual base position interrogated. For example, FIG. 1 illustrates experimental base value distributions for the interrogation of a base at a specific position in a target nucleic acid. Since each interrogation for a particular base will provide base values with respect to all four bases, the lower level base values can be identified by individual base, as in FIG. 1, or the lower base values may be grouped into a single distribution as illustrated in FIG. 2.

For methods in which two bases are interrogated in the sequencing experiments, 16 corresponding measurements can be determined for each of the 16 2-mer sequences.

In one aspect of the present invention, a relative base value for an interrogated nucleotide may be obtained by dividing the obtained actual intensity signal value, preferably without normalization, with the sum of all 4 (or, in the case of 2-mers, 16) actual measurements. Obtaining relative values using this or similar approaches can create comparable base values between target sequences that may have different copy number or other experimental variability. In another aspect of the present invention, different mean or median or other statistical values for each base value can be calculated and compared with the actual target sequence values.

Various approaches can be used to determine the distribution of experimental base values for use in the present invention. One approach is to calculate mean and standard deviation for each individual base value distribution. Another approach is to generate the data used for the creation of the distribution using a histogram of from an approximately 10- to 100-bin histogram. Yet another approach is to rank all relative values (e.g., by percentiles) each individual distribution. An aspect of the process is to assign the highest rank to the smallest value in the values obtained other than those in the top distribution.

### Grouping of Interrogated Nucleotides by Associated Experimental Measurements

In certain aspects of the invention, the experimental base values for individual nucleotides can be used in the methods of the invention to directly determine relative base probabilities for each interrogated nucleotide position. In other aspects of the invention, the use of associated experimental measurements can be used for the initial dividing of the data into groups for further analysis, e.g., determination of more precise distributions of experimental base values for each particular group. It is well within the abilities of those skilled in the art to identify associated experimental measurements from any given sequencing experiment or set of sequencing experiments that can be used in the division and more precise analysis of experimental base values and, as such, an exhaustive list is not provided so as not to obscure the fundamental concepts of the invention. The grouping of the experimental base values is thus described primarily with respect to the use of position context as an associated experimental measurement, although it is intended that the methods of the invention include other associated experimental measurements such as target nucleic acid base content, quantity of target nucleic acid in the sequencing experiment(s), changes in experimental conditions, and the like.

In a preferred aspect of the invention, the ability to use contextual information, such as the identification of one or more other bases in the target sequence that are in a defined position relative to the interrogated nucleic acid, e.g., a base adjacent to an interrogated base, two bases adjacent to an interrogated base, two bases adjacent on either side of the interrogated nucleotide, etc. Such additional bases used in the calling of an interrogation base are referred to herein as “context bases”

In one aspect of the invention, a statistically significant number of experimental base values can be categorized into four or more sequence groups according to the identification of one or more context base. Categorization of experimental base values for specific nucleotide positions can be performed by selecting a base call for the context base(s) with the highest fluorescence intensity as determined by raw data, normalized fluorescence intensity, or other primary identifying measures. The assumption here is that in large majority of the cases the base with the highest intensity is the correct base, and thus the intensity measurement of the context base(s) will be indicative of the identity of the specific base. When normalization of the fluorescent intensity is used to identify the context base(s), the normalization may be performed using known factors from prior experiments, by comparison to reference sequences, or by statistical behavior of data measuring each base. Normalization minimizes intensity differences due to differences introduced by experimental variation, such as the concentration of reagents such as probes or dyes.

To increase the statistical significance and accuracy of the data used in categorization of the nucleotides, a larger number of target sequences queried per sequence group is preferably used to provide more accurate results. Preferably, at least 30 or more individual base experimental base values are included in each group, even more preferably at least 50 or more individual base experimental base values are included in each group, and even more preferably at least 1000 or more individual base experimental base values are included in each group. Each base position interrogated in a target nucleic acid may be in a different group. In the simplest case, each interrogated base is placed in a group specific for that position in the sequencing experiment corresponding to the four bases—in the case of DNA, G, A, T, and C.

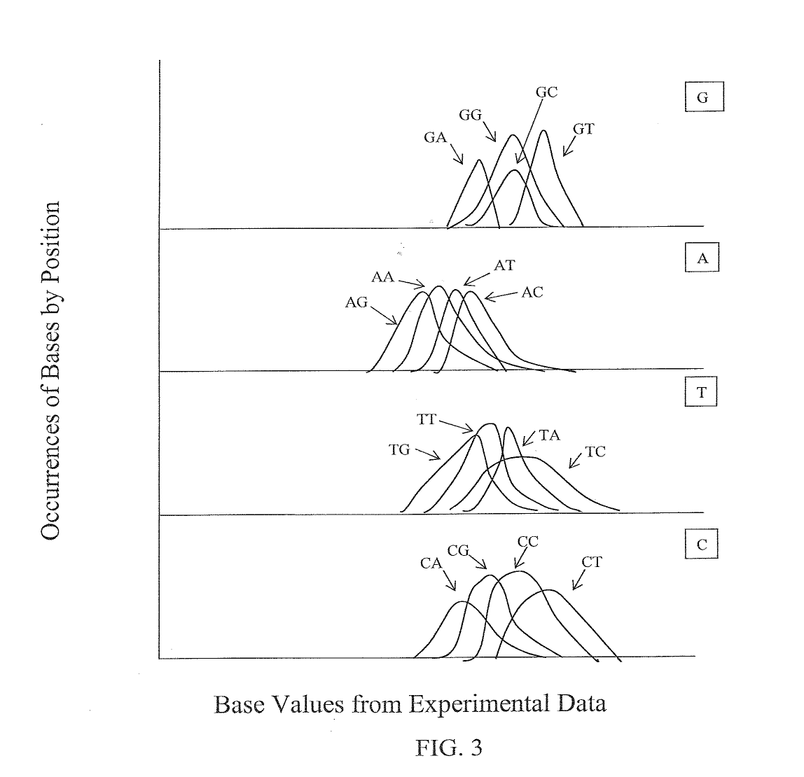

In specific embodiments, however, a further subdivision of target sequences may be performed after forming target groups by the strongest normalized experimental base values of the multiple reads of interrogation bases, such as a categorization into four groups each for G, A, T, and C for each single base read (See FIG. 1). In specific embodiments, each of these four primary groups based on experimental base values for the interrogation base may be further divided into up to 16 final groups according to the strongest base value at a context base, e.g., a context base adjacent to the interrogated base. This further subdivision is demonstrated for the base call with the strongest base value based on the information provided by the context base(s) for each of the four bases in FIG. 3. For clarity, and to avoid obscuring the concepts of the invention, the subdivision of the three bases with lower experimental base values for each position is not shown in the figure.

Subdividing of the four primary groups of experimental base values may also be performed by utilizing the experimental base calling for interrogations in the target sequences and context base information provided by comparison of the target nucleic acid sequence with a reference sequence. If a majority of target nucleic acids are mapped to a reference sequence, and substantially all target sequences that have the best match to that reference sequence, even if they differ in some bases, may be determined to have a sequence identical to that reference sequence. The information provided by these verified sequences are then used for sub-dividing targets into four or more groups per target position. This approach works especially well when there are regions with a high coverage of reads that define correct sequence in spite of quite high error in individual reads.

For sequences that have high target nucleic acid coverage in the sequencing experiment, but which have a sequence-dependent lower signal (e.g. due to consistent lower read quality), the high quality reads that are obtained can be mapped to a reference and their sequences confirmed. In addition, data from sequencing part of one or more adapters linked to targets or sequencing targets from an internal control nucleic acid such as E. coli may be used to create representative groups or to supplement test targets.

Final groups of experimental base values of interrogated nucleic acids may be created to various level of precision based on selected parameters. For example, if 8 bases are interrogated between two adapters (with a read of four bases adjacent to each adapter) using cPAL sequencing (as described above) with 8-mer probes, reading a single base at a time, a preferred signal intensity grouping method is to first form four primary groups (one for each base) for each of 8 positions. Each primary group is then further subdivided according to information provided by interrogation of one or more selected context base(s), e.g., identified highest experimental base values of relevant neighboring sequences.

In one specific aspect using cPal sequencing technology, each primary signal intensity group for interrogating a specific nucleotide position in a target nucleic acid can be subdivided into 256 groups according to other four bases interrogated in the sequencing reaction (context bases) in the first 5 bases next to the adapter or next to ligation site. A very specific example uses a single base A for all 8 positions interrogated—two sets of four primary reads where A is the base with the highest experimental base value. In this example, Bs represent any of the other four context bases used for forming 256 subgroups for each of 8 A-groups, and Ks represent surrounding nucleotides.

KKKKKKKKKKKBBBBBBBBKKKKKKKKKKKKK
ABBBB
BABBB
BBABB
BBBAB
BABBB
BBABB
BBBAB
BBBBA

For this example, to have 1000 targets per final group, 256,000 targets need to be interrogated. Final subdivision based on more or less than four neighboring bases may also be used to subdivide the four primary groups.

Different or further subdivisions may also, in certain circumstances, be beneficial. For example, when a specific experimental bias is identified in the sequencing experiment (e.g., due to differences in fluorescent intensity for different probes used in identification of specific bases), the subdivisions can be determined to take such changes into account. One example is to divide groups of experimental base values for interrogated nucleotides into 2, 3, 4, 5 or even more sub-groups according to one of statistical or actual measures that differentiate targets. One such measure may be median signal of all measured signals for a target nucleic acid. Sub-grouping by target properties may be beneficial because differences in copy number per target nucleic acid may influence response of reagents in the sequencing experiments (e.g., probes, dNTPs).

### Determination of Relative Base Probabilities

Relative base probabilities can be determined by comparing experimental measurements for individual bases in target nucleic acids, and, using one or more distributions calculated from experimental data (e.g., from the same sequencing experiment or a previous sequencing experiment conducted under substantially the same experimental conditions). Each individual interrogated base can be directly compared to a corresponding distributions of measurements for individual nucleotides at specific positions in each of said target nucleic acid groups, and calculating the likelihood (i.e., pseudo probability or pseudo likelihood) of the presence of that base, with or without context base(s) information, at the interrogated position in each target nucleic acid.

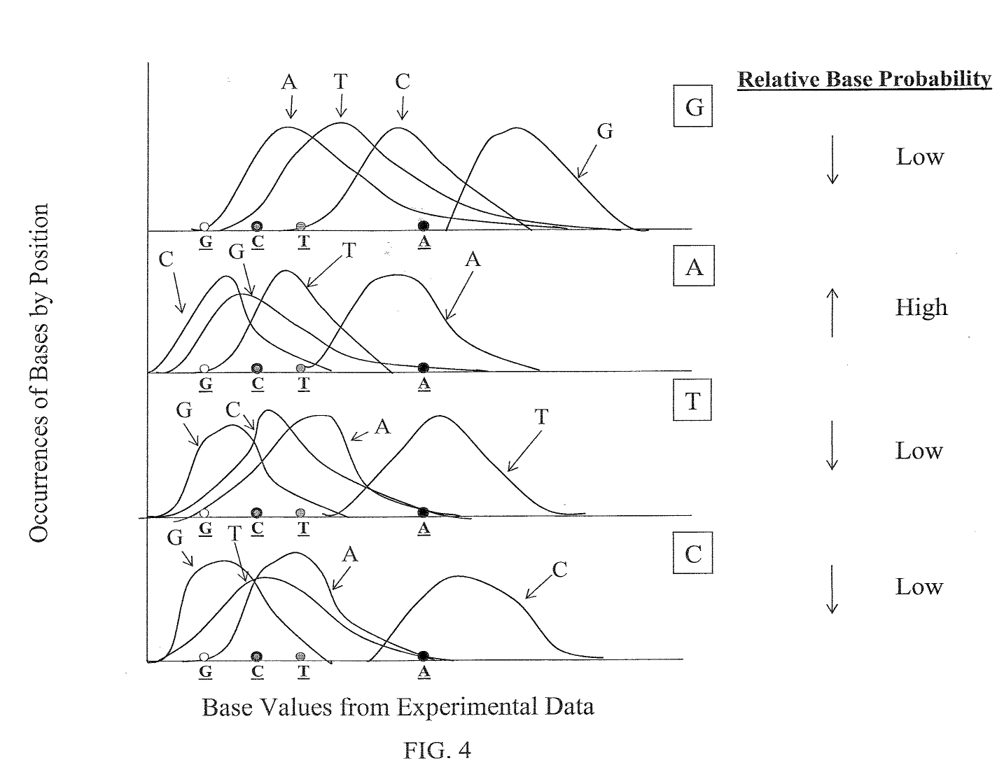

There are various ways to perform these comparisons. Preferably comparisons are performed position by position for each interrogated nucleotide in a given target nucleic acid. For the single base read, there are four measurements for each tested position (See FIG. 4). For the simplest case, of only 4 groups per position, these four measurements are compared separately with each base group to calculate the likelihood that the base at the interrogated position is A, T, C or G at this target at this position. In FIG. 4, the measurements of base A are illustrated as black dots, base C with dark grey dots, base T a light grey dot with a black outline, and G a white dot with a black outline. When, for example in FIG. 4, four different measurements of experimental base values for an interrogated nucleotide are compared, each measurement is compared to the corresponding base distribution for that group to obtain a measure of likelihood that that signal intensity belongs to the distribution for that base. Here, the only measured base value that is within the higher base value distribution is A, which has a measurement that places it at or near the peak value of the distribution; thus, the relative probability of the base being A is high. None of the other measurements fall within the relevant distribution region for their particular base value, and thus the relative probability of the base being T, G, or C is low.

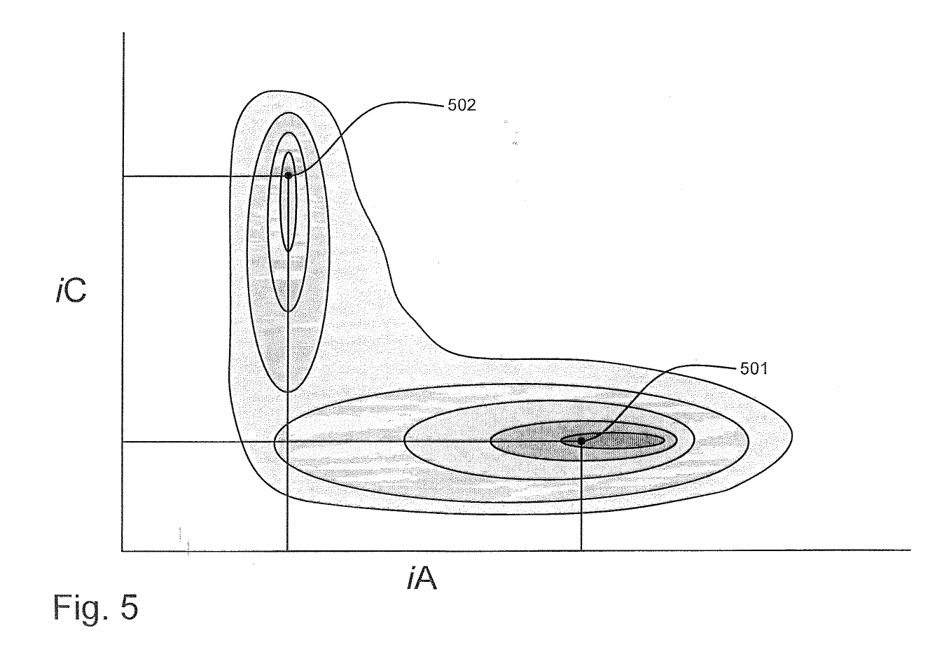

In other specific aspects, rather than analyzing the four potential bases individually for determination of the base value distributions, a base call can be analyzed with relative to two, three or even four bases. An example of this using two bases—C and A—is shown in FIG. 5. The contours represent occurrence levels for each base. An experimental base value (here, a signal intensity created using fluorescence) obtained is analyzed with respect to both A and C, and the relative base probability of this base being either A or C at a position in a target nucleic acid is determined by the position within the intensity graph relative to the positions (i.e., distribution) of A and C values of all other target nucleic acids. Recognition of clusters and definition of their statistical properties can thus be used in determining relative base probabilities.

In another aspect of the invention, an estimate imprecision (“sigma”) of determination of different intensities for each base read can be determined by repeating one cycle twice or using values from prior experiments. This sigma value can also be calculated from finding matching targets from the same or other experiments conducted under substantially similar conditions with proper experimental base value normalization. An estimated imprecision may be used to calculate more accurate base call likelihoods. The estimate of imprecision of base value measure for an interrogated base may also be used to calculate the imprecision in determining confidence calls of each base or sequence variant in the analyzed target sequence

If target subgroups are formed for each base (or two bases) read position (for example sub-groups based on using neighboring bases) there are various ways of defining the likelihood of each base value from the likelihoods of each sub-groups. The highest likelihood value among all sub-groups for each base value can be read by comparison of the obtained values of the experimental base values of a specific interrogation base (or, in the case of using 2-mers for identification, two bases) with the distribution values calculated. Representative likelihood values can also be used to determine specific relative base probabilities from all or specific subgroup values. The final likelihood values calculated for four bases (or 16 2-mer sequences or all longer unit reads) at a given target position may be used to calculate a final normalized probability for 4 bases (or 16 2-mers) at that position or two given positions;

If calculations of probabilities for each base are performed with full dependence (for example, using all 6-8 bases next to an adapter end as context bases), calculation of relative base probabilities for independent interrogation bases are dependent upon initial identification of the greatest base value for each of the context base positions used in the analysis. The context bases used for calculations may be only a single identified base, from between 2-4 identified context bases, or between 3-5 identified context bases. Accurately determined relative base probabilities for each interrogated base can also be used to determine the quality of the specific base calling such data may be used in further analysis, e.g., full-scale assembly of the target nucleic acid.

### Computer Systems for Implementation of the Invention

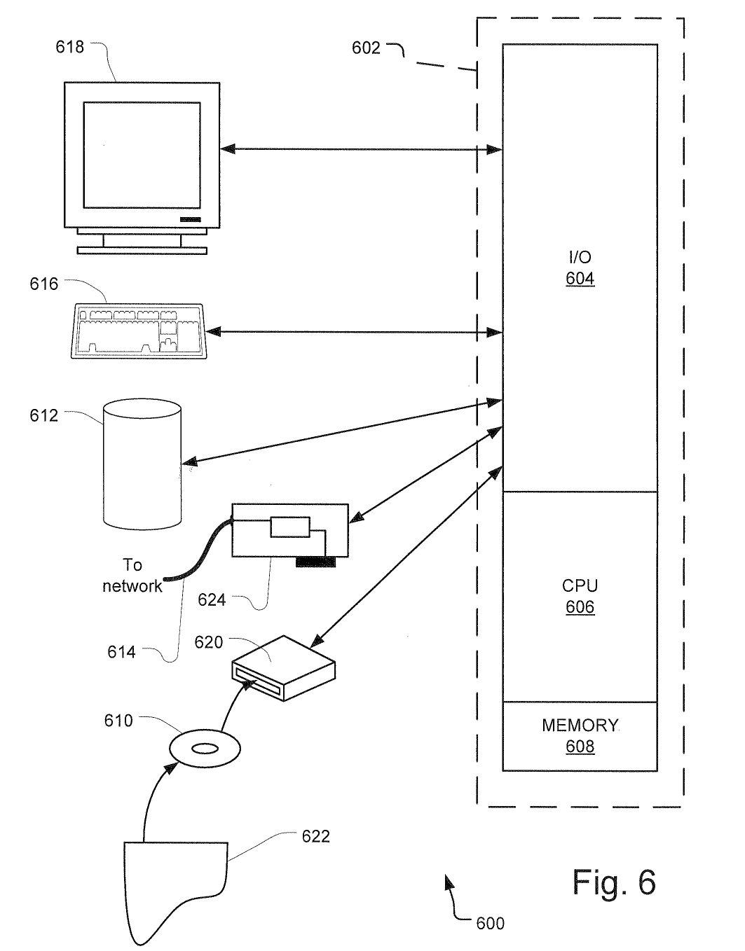

FIG. 6 illustrates an example computing system that can be used to implement the described technology. A general purpose computer system 600 is capable of executing a computer program product to execute a computer process. Data and program files may be input to the computer system 600, which reads the files and executes the programs therein. Some of the elements of a general purpose computer system 600 are shown in FIG. 6 wherein a processor 602 is shown having an input/output (I/O) section 604, a Central Processing Unit (CPU) 606, and a memory section 608. There may be one or more processors 602, such that the processor 602 of the computer system 600 comprises a single central-processing unit 606, or a plurality of processing units, commonly referred to as a parallel processing environment. The computer system 600 may be a conventional computer, a distributed computer, or any other type of computer. The described technology is optionally implemented in software devices loaded in memory 608, stored on a configured DVD/CD-ROM 610 or storage unit 612, and/or communicated via a wired or wireless network link 614 on a carrier signal, thereby transforming the computer system 600 in FIG. 6 to a special purpose machine for implementing the described operations.

The I/O section 604 is connected to one or more user-interface devices (e.g., a keyboard 616 and a display unit 618), a disk storage unit 612, and a disk drive unit 620. Generally, in contemporary systems, the disk drive unit 620 is a DVD/CD-ROM drive unit capable of reading the DVD/CD-ROM medium 610, which typically contains programs and data 622. Computer program products containing mechanisms to effectuate the systems and methods in accordance with the described technology may reside in the memory section 604, on a disk storage unit 612, or on the DVD/CD-ROM medium 610 of such a system 600. Alternatively, a disk drive unit 620 may be replaced or supplemented by a floppy drive unit, a tape drive unit, or other storage medium drive unit. The network adapter 624 is capable of connecting the computer system to a network via the network link 614, through which the computer system can receive instructions and data embodied in a carrier wave. Examples of such systems include Intel and PowerPC systems offered by Apple Computer, Inc., personal computers offered by Dell Corporation and by other manufacturers of Intel-compatible personal computers, AMD-based computing systems and other systems running a Windows-based, UNIX-based or other operating system. It should be understood that computing systems may also embody devices such as Personal Digital Assistants (PDAs), mobile phones, gaming consoles, set top boxes, etc.

When used in a LAN-networking environment, the computer system 600 is connected (by wired connection or wirelessly) to a local network through the network interface or adapter 624, which is one type of communications device. When used in a WAN-networking environment, the computer system 600 typically includes a modem, a network adapter, or any other type of communications device for establishing communications over the wide area network. In a networked environment, program modules depicted relative to the computer system 600 or portions thereof, may be stored in a remote memory storage device. It is appreciated that the network connections shown are exemplary and other means of and communications devices for establishing a communications link between the computers may be used.

In an exemplary implementation, a reference sequence module, a raw data signal intensity module, a refined signal intensity module and other modules may be incorporated as part of the operating system, application programs, or other program modules. Signal intensities, signal intensity distribution, base positions, reference sequence, and other data may be stored as program data in memory 608 or other storage systems, such as disk storage unit 612 or DVD/CD-ROM medium 610.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

What is claimed is:

1. A method for determining a relative base probability, comprising:
(a) providing experimental base values for a base at a position in a statistically significant set of target nucleic acids;
(b) creating a distribution of said experimental base values;
(c) determining a relative base probability of a base at a position of a target nucleic acid by comparing its experimental base value with the distribution of experimental base values.

2. The method of claim 1, wherein the experimental base values are obtained for the same position in a target nucleic acid relative to a priming site or adaptor binding site.

3. The method of claim 1, wherein the method further comprises an adjustment of the experimental base values before creation of said distribution.

4. The method of claim 3, wherein the adjustment is a normalization of experimental base values.

5. The method of claim 1, wherein all experimental base values are obtained in a single sequencing experiment.

6. The method of claim 1, wherein the base probability is determined using multiple experimental base values for one base for a position in the set of target nucleic acids.

7. The method of claim 1, wherein the base probability is determined using multiple experimental base values for all bases for a position in the set of target nucleic acid.

8. The method of claim 7, wherein the base probability is determined for each base for a position in a target nucleic acid.

9. The method of claim 7, wherein four groups of four experimental base value distributions are created.

10. The method of claim 8, wherein the distribution is characterized by clustering.

11. The method of claim 8, wherein the base probabilities are determined for multiple positions in a target nucleic acid.

12. The method of claim 1, wherein the method further comprises:
(d) calling a base at a specific position in the target nucleic acid based on its relative base probability.

13. A method for determining relative base probabilities, comprising:
(a) providing experimental base values for a base at a position in set of target nucleic acids;
(b) dividing said base values into two or more groups according to associated experimental measurements, wherein each group comprises a statistically significant number of experimental base values;
(c) creating a distribution of said bases values for each group of step (b);
(d) determining the relative base probability of a base in a position of a target nucleic in each group by comparing its experimental base value with the distribution of experimental base values in the relevant group.

14. The method of claim 13, wherein the associated experimental measurements comprise experimental base values for one or more other positions within said target nucleic acids.

15. The method of claim 13, wherein the associated experimental measurements comprise the quantity of target nucleic acid analyzed.

16. The method of claim 13, wherein the associated experimental measurements comprise the nucleotide base content of the target nucleic acid.

17. The method of claim 13, wherein the base probability is determined using multiple experimental base values for all bases for a position in the relevant group of target nucleic acids.

18. The method of claim 17, wherein the base probability is determined for each base for a position in a target nucleic acid.

19. The method of claim 13, wherein the distributions of said base values for each group of step (b) are provided by previous or control experiments;

20. The method of claim 13, wherein the method further comprises:
(e) calling a base at a specific position in the target nucleic acid based on its relative base probability.

21. A method of determining a relative base probability in a target nucleic acid, comprising the steps of:
(a) obtaining a plurality of experimental intensity base values at a position in a target nucleic acid;
(b) dividing the experimental intensity values into groups based on the identification of a second base in a target nucleic acid with a known position relative to the first base;
(c) creating an intensity value distribution for each group based on the plurality of base values obtained, wherein the groups comprise statistically significant number of experimental intensity values; and
(d) comparing the experimental intensity value of the first base to the distribution created from a relevant group to determine a relative base probability.

22. A computer program for determining relative base probabilities, comprising:
(a) computer code that receives a plurality of signals corresponding to base values for a target nucleic acid;
(b) computer code for creating a distribution of said experimental base values;
(c) computer code for determining a relative base probability of a base at a position of the target nucleic acid by comparing its experimental base value with the distribution of experimental base values; and
(d) a computer readable medium that stores said computer codes.

23. The program of claim 22, further comprising:
(a) computer code that generates a base call for the base at a position in a target nucleic acid.

24. A system for determining relative base probabilities, comprising:
(a) a processor; and
(b) a computer readable medium coupled to said processor for storing a computer program comprising:
i. computer code that receives a plurality of signals corresponding to a statistically significant number of experimental base values for a target nucleic acid;
ii. computer code for creating a distribution of said experimental base values; and
iii. computer code for determining a relative base probability of a base at a position of the target nucleic acid by comparing its experimental base value with the distribution of experimental base values.

25. The system of claim 24, further comprising:
iv. computer code that generates a base call for the base at a position in a target nucleic acid.
